(19) United States
(12) Patent Application Publication
(10) Pub. No.: US 2006/0173486 A1
(43) Pub. Date: Aug. 3, 2006

(54) Device for staunching postpartum haemorrhage

(76) Inventors: Gerard Burke, Limerick (IE); Paul Anthony Slattery, Limerick (IE)

Correspondence Address: JACOBSON HOLMAN PLLC, 400 SEVENTH STREET N.W., SUITE 600, WASHINGTON, DC, 20004, US

(21) Appl. No.: 11/391,475

(22) Filed: Mar. 29, 2006

Related U.S. Application Data
(63) Continuation of application No. PCT/IE04/00133, filed on Sep. 29, 2004.

(51) Int. Cl. A61M 29/00

(52) U.S. Cl. 606/193

(57) ABSTRACT

A device (1) for staunching postpartum haemorrhage of the uterus (U). The device (1) comprises an inflatable internal balloon (2) having an inlet connected by a fitting (3) over a distal end (4) of a tubular conduit (5). The tubular conduit (5) has a proximal end (6) for connection to an inflation source for manually inflating the internal balloon (2). A non-return valve (8) is fitted in the conduit (5) at the proximal end (6). The device (1) has an inflatable external balloon (10) for indicating when the surface of the internal balloon (2) has engaged with the wall of the uterus (U). The external balloon (10) also acts as an indicator to indicate subsequent contraction of the uterus (U).

The invention relates to a device for use in cases of obstetric haemorrhage following childbirth, known as postpartum haemorrhage (PPH).

Postpartum haemorrhage (PPH) is a common condition affecting up to 5% of births. It is serious in that it is estimated that at least 150,000 women die annually around the world from this condition and the actual number may be far higher.

Immediately following childbirth the uterus begins to contract This contraction gives rise to what is known as the tourniquet effect, whereby the haemorrhaging uterine vasculature is constricted by the tightening muscle fibres and haemostasis is achieved. In some women, roughly 5% , this mechanism is compromised and following childbirth the uterus is atonal and unable to contract Current treatment involves the use of pharmacological agents such as Oxytocin and Prostaglandins. Where these prove to be unsuccessful, hysterectomy may be the only course of action. Hysterectomy is only available in well-staffed and equipped maternity units. Before proceeding with hysterectomy, packing of the uterus with gauze has been attempted but is not now considered effective. The procedure of packing and unpacking the uterus is difficult at best and any packing that is unretrieved leads to serious complications. Furthermore, the gauze becomes saturated with blood and blood loss may be more difficult to detect. There are also concerns about infection.

In developing world situations where access to facilities and pharmacology is poor, the outcome is usually far more serious in that death results. On average, a mother will die within 2 hours of the onset of PPH. Survivors of PPH in these circumstances often face severe Lifetime problems of anaemia and recurrent infections. Towel packing is the only method of treatment currently in use in the developing world. Towels or other bulky absorbent materials are used to pack the uterus in the hope of applying enough pressure to stop the bleeding. This method is usually ineffective and has major problems associated with unretrieved packing and potential infection.

There is therefore a need for a device which will address these serious clinical problems in a cost efficient manner.

### STATEMENTS OF INVENTION

According to the invention there is provided a device for staunching postpartum haemorrhage comprising:
an internal balloon for insertion into a uterus;
the internal balloon being inflatable to engage the wall of a uterus;
a tubular conduit having a distal end in fluid communication with the internal balloon and a proximal end in fluid communication with an inflation source;
the internal balloon being attached to the tubular conduit at a single attachment region to facilitate unconstrained inflation of the internal balloon; and
a first indicator for indicating when the internal balloon has engaged the wall of a uterus.

In one embodiment the device comprises a second indicator for indicating subsequent contraction of a uterus. The first indicator and the second indicator are preferably provided by a single indicator device.

In one case the tubular conduit defines a longitudinal axis, and the internal balloon is inflatable in a direction parallel to the tubular conduit longitudinal axis. Preferably the internal balloon defines three mutually perpendicular co-ordinate axes, and the internal balloon is inflatable in directions parallel to all three co-ordinate axes.

In another case the internal balloon comprises an inlet, and the inlet of the internal balloon is attached to the single attachment region of the tubular conduit. Preferably the internal balloon comprises a continuous balloon wall with a single opening in the balloon wall, the single opening providing the inlet. Ideally an outer surface of the internal balloon is substantially smooth.

In a preferred embodiment the indicator is connected in fluid communication with the tubular conduit. Ideally the tubular conduit comprises an indicator port for connecting the indicator in fluid communication with the tubular conduit. Most preferably the device comprises external valves means intermediate the tubular conduit and the indicator. The indicator may be inflatable. Preferably the indicator comprises an external balloon.

The internal balloon and the external balloon may be configured to facilitate inflation of the internal balloon in the event of a defined fluid pressure within the internal balloon and to resist inflation of the external balloon in the event of the same defined fluid pressure within the external balloon. Preferably the defined pressure is less than or equal to mean arterial pressure.

In one case the dimensions of the internal balloon and the dimensions of the external balloon are configured to facilitate inflation of the internal balloon in the event of a defined fluid pressure within the internal balloon and to resist inflation of the external balloon in the event of the same defined fluid pressure within the external balloon.

Preferably:
r₁/t₁>r₂/t₂
where:
r₁=radius of the internal balloon when in a static uninflated state.
r₂=radius of the external balloon when in a static uninflated state.
t₂=wall thickness of the internal balloon when in a static uninflated state.
t₂=wall thickness of the external balloon when in a static uninflated state.

The ratio of
r₁/t₁:r₂/t₂
may be at least 2:1.

The ratio of
r₁/t₂:r₂/t₂
is preferably approximately 2.5:1.

The volume of the internal balloon when in a static uninflated state is preferably substantially larger than the volume of the external balloon when in a static uninflated state. The ratio of the volume of the internal balloon when in a static uninflated state to the volume of the external balloon when in a static uninflated state may be at least 10:1. The ratio may be at least 15:1. The ratio is approximately 17:1.

The surface area of the internal balloon when in a static uninflated state is preferably substantially larger than the surface area of the external balloon when in a static uninflated state. The ratio of the surface area of the internal balloon when in a static uninflated state to the surface area of the external balloon when in a static uninflated state may be at least 5:1. The ratio is approximately 7:1.

In another case the material properties of the internal balloon and the material properties of the external balloon are configured to facilitate inflation of the internal balloon in the event of a defined fluid pressure within the internal balloon and to resist inflation of the external balloon in the event of the same defined fluid pressure within the external balloon.

Preferably:
E₁<E₂
where:—E₁=modulus of elasticity of the internal balloon;
E₂=modulus of elasticity of the external balloon;

In another embodiment the device comprises a connector for attaching the internal balloon to the tubular conduit Preferably the connector has a proximal region attached to an outer surface of the tubular conduit, and a distal region attached to an outer surface of the internal balloon. Ideally the distal region extends substantially tangentially around at least part of the outer surface of the internal balloon. The connector may taper outwardly from a proximal end of the connector to a distal end of the connector.

In another aspect the invention provides a method of staunching postpartum haemorrhage comprising the steps of:
providing a device comprising an internal balloon, a tubular conduit having a distal end in fluid communication with the internal balloon and a proximal end in fluid connection with an inflation source, and a first indicator;
inserting the internal balloon into a uterine cavity;
inflating the internal balloon until the internal balloon engages the wall of the uterus; and
monitoring the first indicator to determine when the internal balloon has engaged the wall of the uterus.

In one embodiment engagement of the internal balloon with the wall of the uterus causes the first indicator to inflate. Preferably the first indicator is connected in fluid communication with the tubular conduit.

The device may comprise a second indicator, and the method may comprise the step of monitoring the second indicator to determine subsequent contraction of the uterus. Ideally subsequent contraction of the uterus causes the second indicator to inflate. Most preferably the second indicator is connected in fluid communication with the tubular conduit. The first indicator and the second indicator may be provided by a single indicator device.

In one case the method comprises the step of, after engagement of the internal balloon with the wall of the uterus, stopping inflation of the internal balloon.

In another case the method comprises the step of, after engagement of the internal balloon with the wall of the uterus, further inflating the internal balloon to increase contact pressure between the internal balloon and the wall of the uterus. Preferably the method comprises the step of disconnecting tie indicator from the tubular conduit before further inflating the internal balloon.

In a further case the method comprises the step of, after engagement of the internal balloon with the wall of the uterus, further inflating the indicator. Preferably the method comprises the step of at least partially deflating the indicator to further inflate the internal balloon to increase contact pressure between the internal balloon and the wall of the uterus. Ideally the indicator is at least partially deflated by squeezing the indicator.

The postpartum uterus is a large cavity. Because the internal balloon is unconstrained upon inflation, this enables the internal balloon to effectively and quickly expand to fill the volume of the uterus, which can be over 2 litres. When the volume has been filled, the internal balloon engages the wall of the uterus to achieve haemostasis.

The longitudinal inflation is especially important in ensuring that the internal balloon fills the volume of the postpartum uterus to engage the wall of the uterus and achieve haemostasis.

The invention provides a large haemostatic fluid-filled balloon to temporarily pack the uterus and staunch bleeding. The balloon works in two ways. Firstly, direct compression is applied to the placental bed, thus reducing bleeding. Secondly, by stretching the uterine muscle, a reflex contraction may occur encouraging the natural muscle tourniquet effect

The invention provides a large obstetric balloon that can be safely employed as an interim measure before undertaking hysterectomy and furthermore may be usefully employed for transport of bleeding patients to a centre where they can undergo appropriate treatment The balloon can easily be fitted and operated by a paramedical person.

The device provides an indication to the user as to whether or not the balloon is sufficiently expanded and whether or not uterine contraction is occurring. The device cam be used to apply rhythmic stimulation of the uterine muscle, if appropriate.

### BRIEF DESCRIPTION OF THE DRAWINGS

The invention will be more clearly understood from the following description of an embodiment thereof, given by way of example only, with reference to the accompanying drawings; in which:

FIG. 1 is a perspective view of a device for staunching postpartum haemorrhage according to the invention;

FIG. 1(a) is an enlarged, cross-sectional, plan view of a part of the device of FIG. 1; and

FIGS. 2 to 6 illustrate various steps in the use of the device.

### DETAILED DESCRIPTION

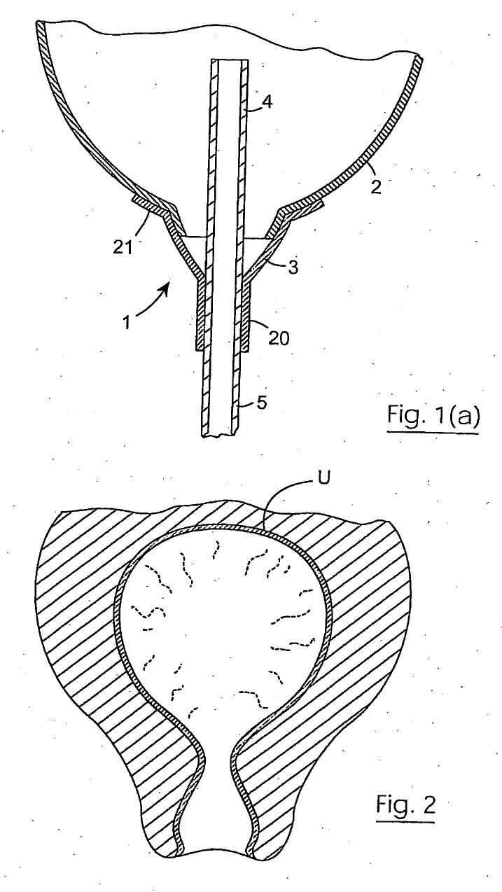

Referring to the drawings there is illustrated a device 1 for staunching postpartum haemorrhage of the uterus U (FIG. 2). The device 1 comprises an inflatable internal balloon 2 having an inlet connected by a fitting 3 over a distal end 4 of a tubular conduit 5 The tubular conduit 5 has a proximal end 6 for connection to an inflation source, preferably a hand-operated pump 7 or a syringe for manually inflating the internal balloon 2. A non-return valve 8 is fitted in the conduit 5 at the proximal end 6. The device 1 has an integral indicator, in this case in the form of an inflatable external balloon 10 for indicating when the surface of the internal balloon 2 has engaged with the wall of the uterus U. The external balloon 10 also acts as an indicator to indicate subsequent contraction of the uterus U.

The internal balloon inlet is attached to die tubular conduit 5 at a single attachment region. This attachment arrangement facilitates unconstrained inflation of the internal balloon 2. In particular the balloon 2 may inflate in all three co-ordinate directions. It is especially important that the balloon 2 can inflate in the direction parallel to the longitudinal axis of the tubular conduit 5 so that the internal balloon 2 can effectively fill the uterine cavity.

The conduit 5 is split adjacent the proximal end 6 thereof and a tee-piece 12 is inserted. The tee-piece 12 has a spur line 13 fitted with a valve 14. On opening of the valve 14, the external balloon 10 is in fluid communication with the internal balloon 2.

The large main or internal balloon 2 is used to directly compress the vascular bed of the uterus U and completely fill the void of the uterus U and thus achieve haemostasis. The indicator balloon 10 indicates when the surface of the main balloon 2 has engaged the wall of the uterus U.

The internal balloon 2 has a continuous, smooth balloon wall with a single opening in the balloon wall which acts as the inlet to the balloon 2. This arrangement ensures that the engagement of the balloon 2 with the wall of the uterus U occurs with the minimum of discomfort/trauma. In particular there are no sharp or protruding parts of the balloon 2 which could act to snag or pierce the wall of the uterus U.

In this system, up to a certain fluid pressure within the balloons 2, 10, which is the mean arterial pressure in this case, the internal balloon 2 inflates, while the external balloon 10 resists inflation. The dimensions of the balloons 2, 10 may be configured to achieve this effect. Alternatively the material properties may be configured to achieve this effect For example the modulus of elasticity of the internal balloon 2 may be lower than the modulus of the elasticity of the external balloon 10.

The balloons 2, 10 are fluidly connected and thus sense the same internal pressure. However, the volume of the internal balloon 2 is several times larger than that of the external balloon 10. Both balloons 2, 10 may be of similar material with similar elastic properties.

The ratio of the balloon static sizes (uninflated) determines the principle of operation of the device. These size ratios may be specifically set to determine the contact pressure that the internal balloon 2 exerts on the wall of the uterus U. In one case the device may deliver contact pressure on the wall of the uterus U equivalent to mean arterial pressure.

Principle of Operation

Stress in a thin-walled spherical vessel is given by the following relationship:
σ=Pr/2t
where P=pressure, r=radius of the vessel, t=wall thickness of the vessel.

The wall thicknesses of the two balloons 2, 10 are identical in this case, the pressure in this fluidically connected system is the same everywhere in the system. Therefore, when the system is pressurised in free space (internal balloon 2 inside the uterus U but not in contact with the wall) it can be seen that the stress exerted on the internal balloon 2 by the fluid is greater than that exerted on the external balloon 10 due to the far greater radius of the internal balloon 2. This forces the internal balloon 2 to expand, while the external balloon 10 remains unexpanded.

On intimate contact with the wall of the uterus U, it is now the uterus U which forms a bouding surface, itself an approximately spherical vessel, with a far more substantial wall thickness. The stress induced in the wall of the uterus U is lower than that in the external balloon 10 and further inflation will cause the external balloon 10 to expand and thus indicate to the clinician contact between the internal balloon 2 and the wall of the uterus U. The system of the internal balloon 2, the external balloon 10 and the uterus U now enters a state of equilibrium.

The system may then be filled with more air, expanding the external sensing balloon 10. The external balloon 10 may then be-squeezed manually to change the contact pressure of the internal balloon 2, thus stimulating the uterine muscle to contract.

An important feature of the device 1 is the difference in size of the balloons 2, 10. The internal balloon 2 is significantly larger than the external balloon 10. The volumetric ratio of the balloons 2, 10 in this system is about 17:1, and the surface area ratio is about 7:1.

As the internal balloon 2 is expanded, the volumetric ratio and the surface area ratio increase and can go to 50:1 or greater.

In this case both the internal balloon 2 and the external balloon 10 are formed from silicone by dip moulding.

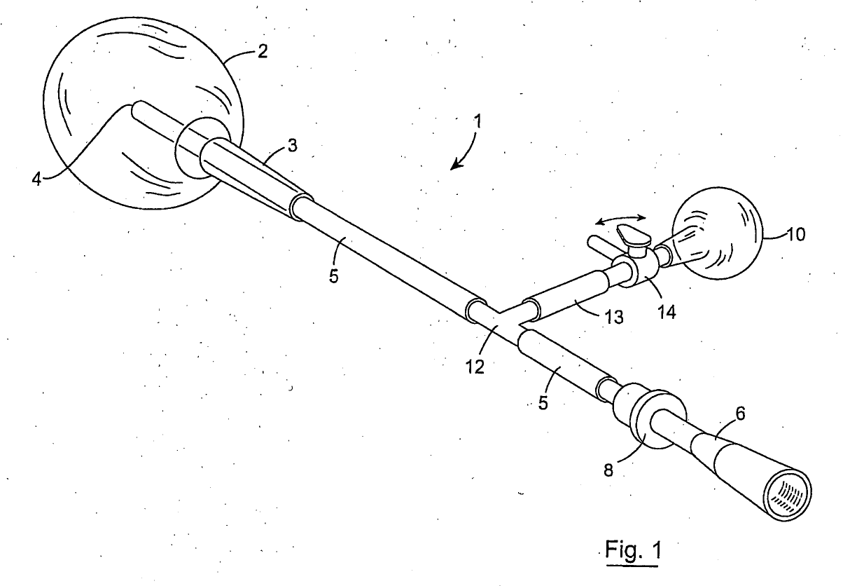

As illustrated in FIG. 1 (a), the connector fitting 3 has a proximal end 20 attached to the outer surface of the tubular conduit 5 and a distal end 21 attached to the outer surface of the balloon 2. The connector fitting 3 tapers outwardly from the proximal end 20 to the distal end 21 in two funnel-type arrangements. The distal end -21 extends tangentially around a part of the balloon 2. It has been found that this type of attachment arrangement results in a more secure attachment. In particular this attachment arrangement is better able to withstand peel stresses than conventional attachment arrangements.

FIG. 2 illustrates a distended atonic uterus U, postpartum.

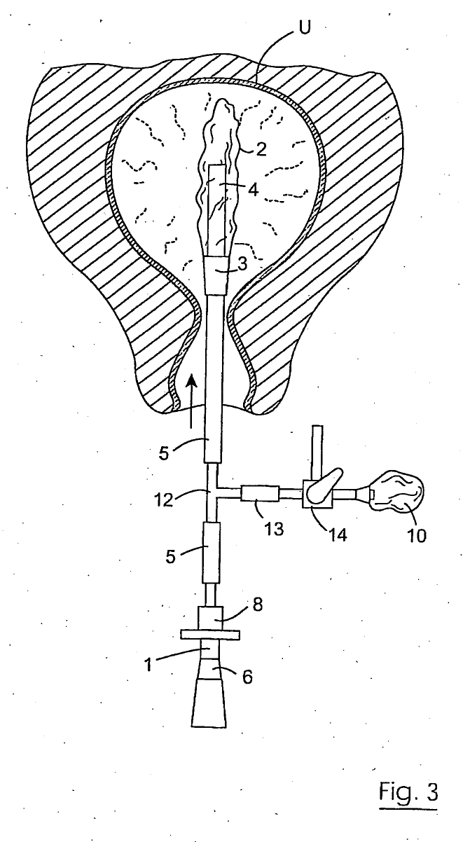

FIG. 3 illustrates the main internal balloon 2 inserted via the vagina and cervix into tie uterus U.

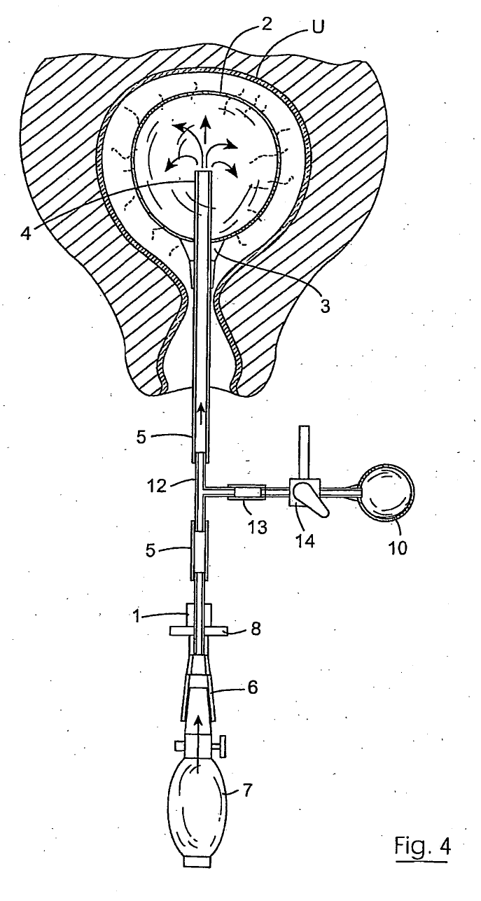

In FIG. 4 the internal balloon 2 is shown being inflated with fluid via a hand pump 7 or syringe. The valve 14 is open to the over pressure external balloon 10 which senses the same pressure as the internal balloon 2.

It will be appreciated that any suitable fluid may be employed for inflation of the internal balloon 2, such as air, water, saline solution, CO₂.

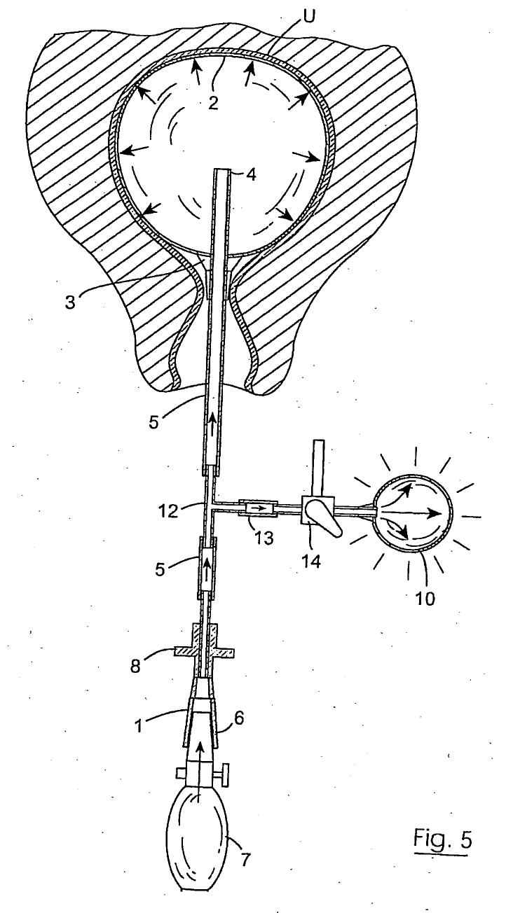

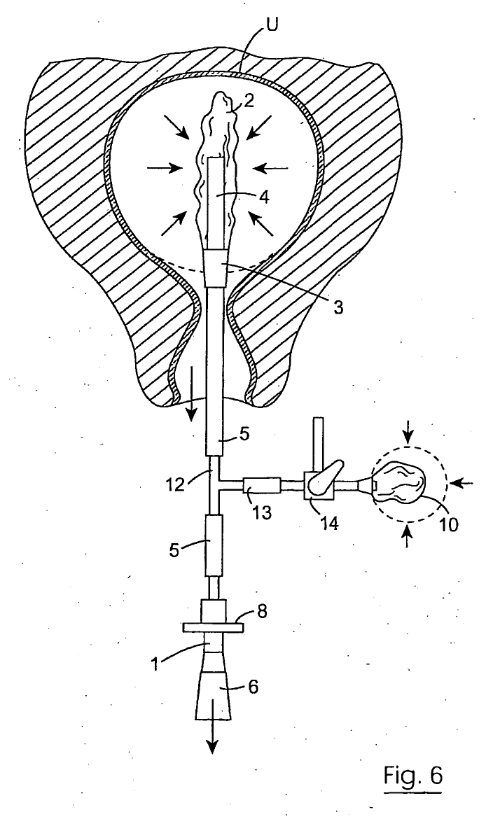

The internal balloon 2 inflates until the outer surface of the internal balloon 2 engages the wall of the uterus U (FIG. 5).

When free and unengaged, the internal balloon 2 is generally spherical 2. Upon engagement with the wall of the uterus U, some conforming of the shape of the internal balloon 2 and the shape of the uterus U occurs until both the internal balloon 2 and the uterus U have conformed to an equilibrium shape. Although the equilibrium shape is illustrated in FIG. 5 as being generally spherical, it will be appreciated that the internal balloon 2 and the uterus U may conform to other non-uniform equilibrium shapes depending on the particular circumstances.

As illustrated in FIG. 5, when the surface of the internal balloon 2 becomes fully engaged with the wall of the uterus U, pressure will rise as more fluid is pumped in, due to the force exerted on the internal balloon 2 by the uterine muscle. The over pressure external balloon 10 will now begin to expand, indicating to the operator that engagement between the internal balloon 2 and the wall of the uterus U has occurred.

The dimensions and material properties of the balloons 2, 10 are chosen to ensure that the external balloon 10 will begin to inflate, when the fluid pressure within the balloons 2, 10 reaches mean arterial pressure, in this case set to a pressure of approximately 100 cm of water.

It will be appreciated that the dimensions and material properties of the balloons 2, 10 may be varied as desired to achieve inflation of the external balloon 10 at any desired pressure. For example, if the device of the invention was applied in veterinary applications, the mean arterial pressure may be higher or lower than 100 cm of water depending on the animal physiology.

It would also be possible to configure the dimensions and material properties of the balloons 2, 10 to achieve inflation of the external balloon 10 at a pressure other than mean arterial pressure. Depending on the particular medical indications, it may be desirable to operate with a pressure higher or lower than the mean arterial pressure in the balloons 2, 10.

The operator may then elect to:
1. Cease inflation.
2. Shut off the valve 14, thus isolating the over pressure external balloon 10 and add a measured volume of fluid to the main internal balloon 2 to boost the contact pressure between the main internal balloon 2 and the uterus wall.
3. Leave the valve 14 open and introduce a volume of fluid so as to expand the over pressure external balloon 10. The expanded over pressure external balloon 10 may be squeezed manually to vary the contact pressure of the main internal balloon 2. This variation in contact pressure may aid the initiation of contraction of the uterine muscle.

The internal balloon 2 may be left ‘in situ’ in the uterus U to maintain haemostasis. It is possible to leave the internal balloon 2 inserted in the uterus U for up to 30 hours, if necessary.

As uterine contraction begins, the over pressure external balloon 10 will expand, indicating that contraction is occurring. Fluid flow may then be varied via the valve 14 keeping the over pressure external balloon 10 small in size and maintaining engagement between the internal balloon 2 and the wall of the uterus U.

The system of the internal balloon 2 connected in fluid communication with the external balloon 10 provides a self-regulating safety mechanism. In particular as the uterus U begins to contract, the external balloon 10 may expand and thus enable the internal balloon 2 to contract to facilitate the contracting of the uterus U. In some cases the uterus U may at least partially relax again. If this should occur, the external balloon 10 may contract and thus enable the internal balloon 2 to expand to maintain engagement with the wall of the uterus U and maintain haemostasis.

### EXAMPLE

A patient presented seven days after a normal delivery with heavy PV bleeding. At examination under anaesthesia, the patient had a retained placental cotyledon and following removal of this, the uterus bled very heavily and became atonic under general anaesthesia. Bleeding was profuse and could not be controlled by Ergometrine, Oxytocin or direct intramyometrial injection of Prostaglandins. The device 1 as described above was inserted and inflated quickly using saline to a volume of 200 cc. The internal balloon 2 was checked for position and fit under ultrasound guidance. There was an immediate and dramatic reduction of bleeding and over the next two hours, the clinician was able to gradually deflate the internal balloon 2 and remove it. There was only minimal blood loss following this.

The internal balloon 2 is inserted into a blind cavity. In a paramedical situation there is not way to determine whether or not a balloon on its own is engaged with the wall of the uterus U with any degree of certainty. The external balloon 10 functions as a detector/regulator of this contact pressure.

The device 1 of the invention can be used for atonic uterus and thus it may prove to be a worthwhile and lifesaving device in countries where there is a high incidence of death from primary postpartum haemorrhage and where access to-surgical facilities and pharmacology is poor.

The device 1 is simple and mobile, and can be used in the field. The device 1 is light and compact and requires no specialised equipment for operation. In particular the device 1 does not require an electrical power source to operate. As such the device 1 is particularly suitable for paramedical applications.

The invention is not limited to the embodiments hereinbefore described, with reference to the accompanying drawings, which may be varied in construction and detail.

1-47. (canceled)

48. A device for staunching postpartum haemorrhage comprising:
an internal balloon for insertion into a uterus;
the internal balloon being inflatable to engage the wall of a uterus;
a tubular conduit having a distal end in fluid communication with the internal balloon and a proximal end in fluid communication with an inflation source;
the internal balloon being attached to the tubular conduit at a single attachment region to facilitate unconstrained inflation of the internal balloon; and
a first indicator for indicating when the internal balloon has engaged the wall of a uterus.

49. The device as claimed in claim 48 wherein the device comprises a second indicator for indicating subsequent contraction of a uterus.

50. The device as claimed in claim.49 wherein the first indicator and the second indicator are provided by a single indicator device.

51. The device as claimed in claim 48 wherein the tubular conduit defines a, longitudinal axis, and the internal balloon is inflatable in a direction parallel to the tubular conduit longitudinal axis.

52. The device as claimed in claim 51 wherein the internal balloon defines three mutually perpendicular co-ordinate axes, and the internal balloon is inflatable in directions parallel to all three co-ordinate axes.

53. The device as claimed in claim 48 wherein the internal balloon comprises an inlet, and the inlet of the internal balloon is attached to the single attachment region of the tubular conduit.

54. The device as claimed in claim 53 wherein the internal balloon comprises a continuous balloon wall with a single opening in the balloon wall, the single opening providing the inlet.

55. The device as claimed in claim 48 wherein an outer surface of the internal balloon is substantially smooth.

56. The device as claimed in claim 48 wherein the indicator is connected in fluid communication with the tubular conduit.

57. The device as claimed in claim 56 wherein the tubular conduit comprises an indicator port for connecting the indicator in fluid communication with the tubular conduit.

58. The device as claimed in claim 56 wherein the device comprises an external valve intermediate the tubular conduit and the indicator.

59. The device as claimed in claim 56 wherein the indicator is inflatable.

60. The device as claimed in claim 59 wherein the indicator comprises an external balloon.

61. The device as claimed in claim 60 wherein the internal balloon and the external balloon are configured to facilitate inflation of the internal balloon in the event of a defined fluid pressure within the internal balloon and to resist inflation of the external balloon in the event of the same defined fluid pressure within the external balloon.

62. The device as claimed in claim 61 wherein the defined pressure is less than or equal to mean arterial pressure.

63. The device as claimed in claim 61 wherein the dimensions of the internal balloon and the dimensions of the external balloon are configured to facilitate inflation of the internal balloon in the event of a defined fluid pressure within the internal balloon and to resist inflation of the external balloon in the event of the same defined fluid pressure within the external balloon.

64. The device as claimed in claim 63 wherein:
r₁/t₁>r₂/t₂
where:
r₁=radius of the internal balloon when in a static uninflated state.
r₂=radius of the external balloon when in a static uninflated state.
t₁=wall thickness of the internal balloon when in a static uninflated state.
t₂=wall thickness of the external balloon when in a static uninflated state.

65. The device as claimed in claim 64 wherein the ratio of
r₁/t₁: r₂/t₂
is at least 2:1.

66. The device as claimed in claim 65 wherein the ratio of
r₁/t₁: r₂/t₂
is approximately 2.5:1.

67. The device as claimed in claim 63 wherein the volume of the internal balloon when in a static uninflated state is substantially larger than the volume of the external balloon when in a static uninflated state.

68. The device as claimed in claim 67 wherein the ratio of the volume of the internal, balloon when in a static uninflated state to the volume of the external balloon when in a static uninflated state is at least 10:1.

69. The device as claimed in claim 68 wherein the ratio is at least 15:1.

70. The device as claimed in claim 67 wherein the ratio is approximately 17:1.

71. The device as claimed in claim 63 wherein the surface area of the internal balloon when in a static uninflated state is substantially larger than the surface area of the external balloon when in a static uninflated state.

72. The device as claimed in claim 71 wherein the ratio of the surface area of the internal balloon when in a static uninflated state to the surface area of the external balloon when in a static uninflated state is at least 5:1.

73. The device as claimed in claim 72 wherein the ratio is approximately 7:1.

74. The device as claimed in claim 61 wherein the material properties of the internal balloon and the material properties of the external balloon are configured to facilitate inflation of the internal balloon in the event of a defined fluid pressure within the internal balloon and to resist inflation of the external balloon in the event of the same defined fluid pressure within the external balloon.

75. The device as claimed in claim 74 wherein:
E₁<E₂
where:—E₁=modulus of elasticity of the internal balloon;
E₂=modulus of elasticity of the external balloon;

76. The device as claimed in claim 48 wherein the device comprises a connector for attaching the internal balloon to the tubular conduit.

77. The device as claimed in claim 76 wherein the connector has a proximal region attached to an outer surface of the tubular conduit, and a distal region attached to an outer surface of the internal balloon.

78. The device as claimed in claim 77 wherein the distal region extends substantially tangentially around at least part of the outer surface of the internal balloon.

79. The device as claimed in claim 77 wherein the connector tapers outwardly from a proximal end of the connector to a distal end of the connector.

80. A method of staunching postpartum haemorrhage comprising the steps of:
providing a device comprising an internal balloon, a tubular conduit having a distal end in fluid communication with the internal balloon and a proximal end in fluid connection with an inflation source, and a first indicator;
inserting the internal balloon into a uterine cavity;
inflating the internal balloon until the internal balloon engages the wall of the uterus; and
monitoring the first indicator to determine when the internal balloon has engaged the wall of the uterus.

81. The method as claimed in claim 80 wherein engagement of the internal balloon with the wall of the uterus causes the first indicator to inflate.

82. The method as claimed in claim 81 wherein the first indicator is connected in fluid communication with the tubular conduit.

83. The method as claimed in claim 80 wherein the device comprises a second indicator, and the method comprises the step of monitoring the second indicator to determine subsequent contraction of the uterus.

84. The method as claimed in claim 83 wherein subsequent contraction of the uterus causes the second indicator to inflate.

85. The method as claimed in claim 84 wherein the second indicator is connected in fluid communication with the tubular conduit.

86. The method as claimed in claim 83 wherein the first indicator and the second indicator are provided by a single indicator device.

87. The method as claimed in claim 80 wherein the method comprises the step of, after engagement of the internal balloon with the wall of the uterus, stopping inflation of the internal balloon.

88. The method as claimed in claim 80 wherein the method comprises the step of, after engagement of the internal balloon with the wall of the uterus, further inflating the internal balloon to increase contact pressure between the internal balloon and the wall of the uterus.

89. The method as claimed in claim 88 wherein the method comprises the step of disconnecting the indicator from the tubular conduit before further inflating the internal balloon.

90. The method as claimed in claim 80 wherein the method comprises the step of, after engagement of the internal balloon with the wall of the uterus, further inflating the indicator.

91. The method as claimed in claim 90 wherein the method comprises the step of at least partially deflating the indicator to further inflate the internal balloon to increase contact pressure between the internal balloon and the wall of the uterus.

92. The method as claimed in claim 91 wherein the indicator is at least partially deflated by squeezing the indicator.
